(19) United States
(12) Patent Application Publication
(10) Pub. No.: US 2022/0202984 A1
(43) Pub. Date: Jun. 30, 2022

(54) GERMICIDAL DEVICES AND APPLICATIONS OF SAME

(71) Applicant: UL Med Inc., Norcross, GA (US)

(72) Inventors: Wenwei Zhang, Norcross, GA (US); Wei Li, Norcross, GA (US); Yuan Di, Norcross, GA (US); Annie You, Norcross, GA (US); Low Wee Fong, Singapore (SG)

(21) Appl. No.: 17/695,112

(22) Filed: Mar. 15, 2022

Related U.S. Application Data
(63) Continuation-in-part of application No. 17/153,191, filed on Jan. 20, 2021.
(60) Provisional application No. 63/180,226, filed on Apr. 27, 2021.
(60) Provisional application No. 62/990,142, filed on Mar. 16, 2020.

(30) Foreign Application Priority Data
Apr. 15, 2021 (CN) 202120765573.9

(51) Int. Cl. A61L 9/22; A61L 9/20; A61L 2/10; A61L 2/24; A61L 2/26

(52) U.S. Cl. CPC A61L 9/22; A61L 9/20; A61L 2/10; A61L 2/24; A61L 2/26; A61L 2209/111; A61L 2202/25; A61L 2209/12; A61L 2209/134; A61L 2209/15; A61L 2202/11; A61L 2202/121; A61L 2209/212

(57) ABSTRACT

A germicidal device includes a purification/disinfection and LED lighting member configured to generate UVC light and negative air ions for destroying microorganisms and removing odors in a space in which the purification and sterilization member is placed; a sensing member configured to detect at least one event occurred in said space in real time; and a microcontroller unit coupled with the purification and sterilization member and the sensing member for controlling operations of the purification and sterilization member in a respective state in accordance with the at least one event occurred in said space and energy saving. The UVC light has one or more wavelengths in a range of about 100-400 nm.

### CROSS-REFERENCE TO RELATED PATENT APPLICATIONS

This application claims priority to and the benefit of U.S. Provisional Patent Application Ser. No. 63/180,226, filed Apr. 27, 2021, which is incorporated herein in its entirety by reference.

This application also claims priority to and benefit of Chinese Patent Application No. 202120765573.9, filed Apr. 15, 2021, which is incorporated herein in its entirety by reference.

This application is also a continuation-in-part application of U.S. patent application Ser. No. 17/153,191, filed Jan. 20, 2021, which itself claims priority to and the benefit of U.S. Provisional Patent Application Ser. No. 62/990,142, filed Mar. 16, 2020, which are incorporated herein in their entireties by reference.

### FIELD OF THE INVENTION

This invention relates generally to purification and sterilization plus LED lighting, and more particularly, to a novel NAIs and UVC plus LED lighting germicidal device that combines smart technology/space/surface and air purification and sterilization functions with or without ozone.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

Air purification refers to providing overall solutions for various indoor environmental problems such as sterilization, dust reduction, haze removal, removal of harmful decoration residues and odors, improving living and office conditions, and enhancing physical and mental health. Indoor environmental pollutants and sources of pollution mainly include radioactive gases, germs, particulate matter, decoration residues, second-hand smoke, etc. Conventionally, the way to purify the air is the use of air purification devices to remove soot particles and germs in the air and surfaces. There exist many types of air purification devices in the market, such as air filter type and UVC (ultraviolet with C wavelength range) type. The existing air purification devices utilize filters and/or UVC to cracking the DNA and RNA molecular chains of germs including viruses so as to kill the germs.

For example, when an UVC air purification device is in use, air is introduced into a filter screen, and then the filter screen is irradiated by the UVC light generated by a UVC generator, thereby killing germs, viruses, etc. on the filter screen. Since UVC can cause damage to human skin, eyes and respiratory tract, it cannot be directly exposed to the external environment, which limits the purification and sterilization efficiency of air purification and sterilization device, and makes it impossible to directly sterilize the indoor environment without dead ends. When there are many germs and viruses in the air and surface, because the air is always in a state of circulation in the indoor environment, germs may exist on various objects in the room. When people come into contact with these objects, it may cause bacterial or virus infection, which will affect people's health; at the same time, when the air purification and sterilization device is working, it will generate a lot of noise, and the filter needs to be replaced regularly. Therefore, the quality of life is lowered and usage costs are increased, as well as environmental pollution.

At present, the purification and sterilization of medium and large places such as theaters, airports, railway stations, warehouses, etc., and public transportation such as school buses, buses, ambulances, airplanes, etc., and logistics transportation such as material transport ships, vehicles, and transport aircraft, mostly use spraying. Disinfection with other disinfectants requires a large amount of disinfectants, and cannot effectively purify and disinfect germs in the air and surface. All these scenarios need to be equipped with lighting device, so the development and design of a suitable intelligent lighting device, while lighting, provides the function of air purification and sterilization, which can effectively make up for the technical problems of air purification and sterilization.

Therefore, a heretofore unaddressed need exists in the art to address the aforementioned deficiencies and inadequacies.

### SUMMARY OF THE INVENTION

In one aspect, this invention relates to a germicidal device. In one embodiment, the germicidal device includes a purification and sterilization member configured to generate UVC light and negative air ions (NAIs) for destroying microorganisms and removing odors in a space in which the purification and sterilization member is placed; a sensing member configured to detect at least one event occurred in said space in real time; and a microcontroller unit (MCU) coupled with the purification and sterilization member and the sensing member for controlling operations of the purification and sterilization member in a respective state in accordance with the at least one event occurred in said space. The UVC light has one or more wavelengths in a range of about 100-400 nm. The at least one event includes a movement in said space, an acoustic wave in said space, an intrusion into said space, an expiration of an on-state time period in which the UVC light is generated, an expiration of an off-state time period in which the UVC light is not generated, and/or an instruction from a remote operation. In addition, the germicidal device may also have a lighting member including one or more LEDs for emitting normal visible light for lighting. The germicidal device therefore is capable of generating NAIs and UVC light for purification and sterilization, and emitting normal visible light for lighting.

In one embodiment, the germicidal device further comprises a housing structure comprises a base, a top cover, support rods connecting the base and the top cover, a casing detachably attached to the base and the top cover, at least one air inlet formed on the base, and at least one air outlet formed on the top cover, wherein the purification and sterilization member is fixed on the base so as to be enclosed by the housing structure.

In one embodiment, the at least one air inlet and the at least one air outlet are arranged on a same vertical plane.

In one embodiment, the casing comprises a plurality of shutter boards that are movably jointed or fixed to each other.

In one embodiment, the casing is operably switchable in an open state or a close state, wherein in the open state the shutter boards are moved to define one or more windows for allowing the UVC light emitted from the lighting member to pass through the one or more windows to illuminate said space, wherein in the close state the shutter boards are moved to close the one or more windows for circulating air at least one air pass-through channel defined between the at least one air inlet and the at least one air outlet.

In one embodiment, the germicidal device is configured to selectively operate in a light disinfection mode in which the casing is in the open state, or in an air purification mode in which the casing is in the close state.

In one embodiment, the germicidal device further comprises a circulating member that is located directly above the purification and sterilization member for operably circulating air.

In one embodiment, the circulating member comprises one or more fans.

In one embodiment, the purification and sterilization member is configured to generate the UVC light has a wavelength of about 185 nm, 222 nm or 253.7 nm, or two wavelengths with one at about 185 nm and the other at about 222 nm or 253.7 nm.

In one embodiment, the purification and sterilization member an NAI generator configured to generate the NAIs, and a lighting member configured to generate the UVC light.

In one embodiment, the lighting member comprises one or more UVC light-emitting diodes (LEDs), an ozone-free UVC lamp, an ozone UVC lamp, or a low-pressure mercury lamp.

In one embodiment, the sensing member comprises a built-in sensor unit comprising at least one radar sensor for detecting a moving or stationary object in said space, at least one motion sensor for detecting a movement in said space, at least one acoustic sensor for detecting an acoustic wave in said space, at least one timer for timing the operation state of the lighting member, at least one temperature sensor for detecting temperature and/or temperature change of said space, or a combination of them.

In one embodiment, the at least one motion sensor comprises one or more microwave sensors, and/or one or more passive infrared (PIR) sensors.

In one embodiment, the built-in sensor unit further comprises a radio frequency (RF) receiver coupled with the MCU for operably detecting the instruction from the remote operation of the remote control, and/or a wireless networking module coupled with the MCU for operably detecting the instruction from the remote operation of the APP of the mobile device.

In one embodiment, the wireless networking module comprises a Bluetooth® module, and/or a Wi-Fi/or a Voice control module.

In one embodiment, the sensing member further comprises a remote sensor unit including an entrance sensor, a photo eye system, or an electric fencing system for detecting an intrusion into said space.

In one embodiment, the germicidal device further comprises a power module for providing power to the lighting member, the sensing member and the MCU.

In one embodiment, the germicidal device further comprises at least one light reflection member configured to collect the UVC light, and at least one grating member configured to direct the collected UVC light into a particular direction to sterilize and purify the entire surface and space.

In one embodiment, the germicidal device is configured to operate in one of:

a default operation mode in which the purification and sterilization member is automatically in an on-state when the time is in an on-state time period, or in an off-state when the time is in an off-state time period, wherein the on-state time period and the off-state time period are predetermined, and wherein the purification and sterilization member emits the UVC light in the on-state, or no light in the off-state;

a manual operation mode having an on-operation and an off-operation, wherein the on-operation causes the lighting member to be in the on-state, and the off-operation causes the lighting member to be in the off-state; and

a remote operation mode in which the purification and sterilization member is operably in one of the on-state and the off-state in response to operations of a remote control or executions of an APP of a mobile device.

In one embodiment, the germicidal device is configured such that when the purification and sterilization member is in the on-state, one of a movement in said space, an expiration of the on-state time period and a remote instruction to turn off the purification and sterilization member detected by the sensing member causes the MCU to switch the purification and sterilization member from the on-state to the off-state; or when the lighting member is in the off-state, an expiration of the off-state time period or a remote instruction to turn on the purification and sterilization member detected by the sensing member causes the MCU to switch the purification and sterilization member from the off-state to the on-state.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows schematically a germicidal device according to one embodiment of the invention.

FIG. 2 show schematically a block diagram of a germicidal device according to one embodiment of the invention.

FIG. 3 shows schematically a front view of a germicidal device according to one embodiment of the invention.

FIG. 4 shows schematically another view of the germicidal device shown in FIG. 3.

FIG. 5 shows schematically a block diagram of a control system of a germicidal device according to one embodiment of the invention.

FIG. 6 shows schematically a germicidal device according to one embodiment of the invention.

FIG. 7 shows schematically the germicidal device of FIG. 6 attached onto a post or wall according to one embodiment of the invention.

FIG. 8 shows schematically a germicidal device according to another embodiment of the invention.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present there between. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this invention, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present invention, and will not be interpreted in an idealized or overly formal sense unless expressly so defined herein.

As used herein, the phrase at least one of A, B, and C should be construed to mean a logical (A or B or C), using a non-exclusive logical OR. As used herein, the term “and/or” includes any and all combinations of one or more of the associated listed items.

As used herein, the term module may refer to, be part of, or include an Application Specific Integrated Circuit (ASIC); an electronic circuit; a combinational logic circuit; a field programmable gate array (FPGA); a processor (shared, dedicated, or group) that executes code; other suitable hardware components that provide the described functionality; or a combination of some or all of the above, such as in a system-on-chip. The term module may include memory (shared, dedicated, or group) that stores code executed by the processor.

As used herein, the terms chip or computer chip generally refers to a hardware electronic component, and may refer to or include a small electronic circuit unit, also known as an integrated circuit (IC), or a combination of electronic circuits or ICs.

As used herein, the term microcontroller unit or its acronym MCU generally refers to a small computer on a single IC chip that can carry out instructions of computer programs by performing basic arithmetic, logical, control and input/output (I/O) operations specified by the instructions for controlling other devices or machines. A microcontroller unit contains one or more central processing unit including one or more processors along with memory and programmable I/O peripherals, and is usually designed for embedded applications.

As used herein, the term system on a chip or its acronym SoC generally refers to an integrated circuit that integrates all or most components of a computer or other electronic system. These components include, but are not limited to, a central processing unit, memory, hard-disk and USB connectivity, secondary storage, input/output ports, and/or their controllers, often alongside other components such as sensing/detecting modules, power modules, and wireless modules (e.g., Wi-Fi and cellular network radio modems) on a single substrate or microchip. It may contain digital, analog, and mixed-signal processing functions.

The term interface, as used herein, generally refers to a communication tool or means at a point of interaction between components for performing wired or wireless data communication between the components. Generally, an interface may be applicable at the level of both hardware and software, and may be uni-directional or bi-directional interface. Examples of physical hardware interface may include electrical connectors, buses, ports, cables, terminals, and other I/O devices or components. The components in communication with the interface may be, for example, multiple components or peripheral devices of a computer system.

The term code, as used herein, may include software, firmware, and/or microcode, and may refer to programs, routines, functions, classes, and/or objects. Some or all code from multiple modules may be executed using a single (shared) processor. In addition, some or all code from multiple modules may be stored by a single (shared) memory. Further, some or all code from a single module may be executed using a group of processors. Moreover, some or all code from a single module may be stored using a group of memories.

The devices and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system. By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more example embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

In one aspect, this invention relates to a novel ultraviolet (UV) germicidal device that is an intelligently controlled lighting purification and sterilization device. The germicidal device utilizes short-wavelength ultraviolet (ultraviolet C or UVC) light to kill or inactivate microorganisms by destroying nucleic acids and disrupting their DNA, which leaves them unable to perform vital cellular functions. The germicidal device also utilizes negative air ions (NAIs) to remove odors and dust in air so as to achieve high-efficiency air purification effect. Meanwhile, after the germicidal device continues working, it can also effectively increase the content of negative ions and oxygen, and further improve the air quality. In addition, the UVC light can kill the pathogens in the air and surface within the effective range without dead ends, so that the indoor objects can be effectively sterilized and disinfected, thereby achieving efficient purification and sterilization. With the functions of remote intelligent control of LED lights and UVC switches, as well turning off the lights when a person comes in and turning on the lights when the person leaves, the germicidal device combines smart technology and surface, space and air sterilization functions with or without ozone, and can be conveniently managed with energy saving.

The germicidal device can be used in a variety of applications, such as indoor (home, dormitory, classroom, theaters, warehouses, etc.) and outdoor (airports, railway stations, etc.) air sterilization, disinfection of elevators, cars, devices such as mobile phones and tablets, appliance such as refrigerators and washers, furniture, and food, water purification, etc., and public transportation such as school buses, buses, ambulances, airplanes, and logistics transportation such as material transport ships, vehicles, and transport aircraft, and so on. The novel germicidal device, among other things, is currently commercialized under the trademark, Inoculight®.

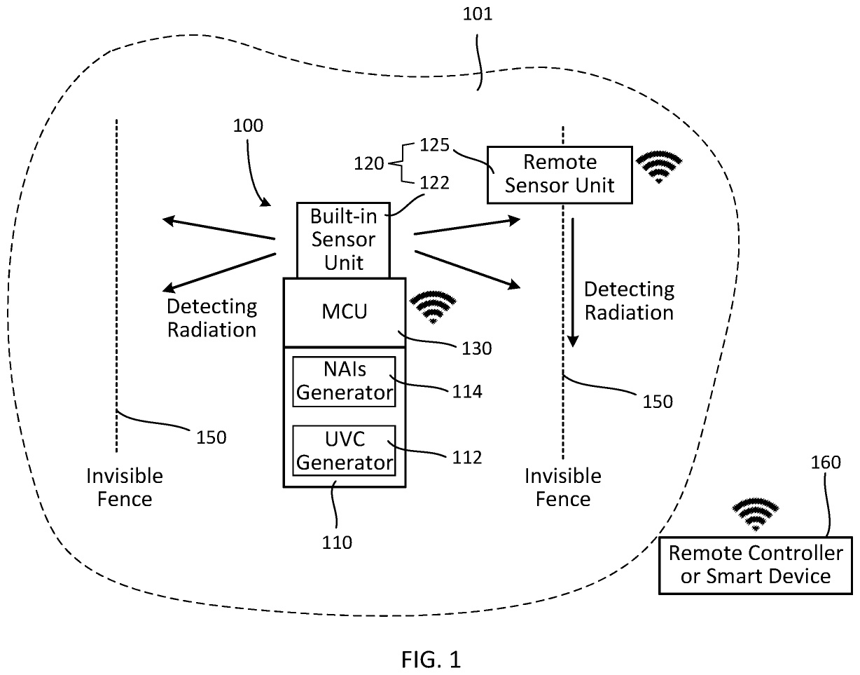

Referring to FIG. 1, a germicidal device 100 is schematically shown according to one embodiment of the invention. The germicidal device 100 is placed in an area (space) 101. The area 101 can be, but is not limited to, a room, office, hallway, or elevator. In this exemplary embodiment, the germicidal device 100 includes a purification and sterilization member 110 configured to emit UVC light for killing or inactivating microorganisms, such as bacteria, fungi, viruses, spores, and/or and other pathogens, and generate negative air ions (NAIs) for removing odors and dust, in the area 101. In one embodiment shown in FIG. 1, the purification and sterilization member 110 has a lighting member (e.g., UVC generator) 112 configured to generate the UVC light and an NAI generator 114 configured to generate the NAIs. The UVC light includes the one or more wavelengths in a wavelength range of about 10-400 nm, preferably about 150-280 nm. In one embodiment, the UVC light has a wavelength of about 185 nm, 222 nm or 253.7 nm, or dual wavelengths with one at about 185 nm and the other at about 222 nm or 253.7 nm. The UVC light is used with or without addition of Ozone.

The lighting member 112 is operably in an on-state in which the lighting member 112 emits the UVC light for killing or inactivating the microorganisms in the area 101, or in an off-state in which the lighting member 112 emits no light.

In addition, the lighting member 112 may include first LEDs for operably emitting UVC light for purification and sterilization, and second LEDs for emitting normal visible light for lighting. As such, when in the on-state, the first LEDs of the lighting member 112 emits the UVC light for killing or inactivating the microorganisms in the area 101, and when in the off-state, the first LEDs of the lighting member 112 emits no light, while the second LEDs of the lighting member 112 emits normal visible light for lighting. Optionally, the second LEDs of the lighting member 112 can also emit normal visible light for lighting when in the on-state.

The germicidal device therefore is capable of generating NAIs and UVC light for purification and sterilization, and emitting normal visible light for lighting. In some embodiments, the germicidal device is a three-in-one device with smart control, which can operably generate NAIs, UVC light, and normal visible light, as desired.

In some embodiments, the lighting member 112 includes a low-pressure mercury lamp that utilizes low pressure (<10⁻² Pa) excited mercury vapor to emit the UVC light, which has two main spectral lines: one is 253.7 nm or quasi-molecular discharging generated 222 nm wavelength; the other is 185 nm wavelength. Both spectral lines are invisible to naked eyes. The UVC light of the 253.7 nm or 222 nm wavelength can sterilize surface, while the 185 nm wavelength light can ionize the air to produce ozone. The difference between these two UVC light lamps is whether they produce ozone or not. Ozone is also highly oxidizing and efficient sterilization (much higher than UV efficiency), which can remedy the shortcomings of the UVC light because of its rectilinear propagation creating a blind/dead spot in disinfection. However, ozone may not be suitable for certain occasions due to its smell. The two kinds of lamps are mainly different in the process of lamp materials. The ozone-free lamp is made by adding a 185 nm transmission barrier material in the lamp tube, so that the 185 nm wavelength of the UVC light cannot be transmitted. Accordingly, there is no 185 nm wavelength light on the surface of the lamp tube to excite oxygen in the air, and no ozone is produced.

In some embodiments, the lighting member 112 includes one or more UVC light-emitting diodes (LEDs). It should be appreciated that other types of UVC light sources can also be utilized to practice the invention.

In some embodiments, the NAI generator 114 is configured to generate the NAIs once the germicidal device 100 is powered on. That is, the NAI generator 114 can still generate the NAIs even the lighting member 112 is operably in the off-state. After the germicidal device is powered on or continues working, the NAI generator 114 can effectively increase the content of negative ions and oxygen, which further improve the air quality. In some embodiments, the NAI generator 114 is configured to generate the NAIs from time to time, i.e., generate the NAIs during predetermined time periods. Alternatively, the NAI generator 114 can be configured to generate the NAIs when the lighting member 112 is operably in the on-state, and no NAIs when the lighting member 112 is operably in the off-state, respectively.

As shown in FIG. 1, the germicidal device 100 also includes a sensing member 120 for operably detecting at least one event occurred in the area 101 and a microcontroller unit (MCU) 130 coupled with the purification and sterilization member 110 and the sensing member 120 for controlling operations of the purification and sterilization member 110, i.e., the lighting member 112, in a respective state in accordance with the at least one event occurred in the area 101. In certain embodiments, the at least one event includes, but is not limited to, a movement in the area 101, an acoustic wave in the area 101, an intrusion into the area 101, an expiration of an on-state time period in which the lighting member 112 is in the on-state, an expiration of an off-state time period in which the lighting member 112 is in the off-state, and/or an instruction from a remote operation 160.

For example, when the lighting member 112 operates in the on-state, one of a movement in the area 101, an expiration of the on-state time period and a remote instruction to turn off the lighting member 112 detected by the sensing member 120 causes the MCU 130 to switch the lighting member 112 from the on-state to the off-state. When the lighting member 112 operates in the off-state, an expiration of the off-state time period or a remote instruction to turn on the lighting member 112 detected by the sensing member 120 causes the MCU 130 to switch the lighting member 112 from the off-state to the on-state.

In some embodiments, the sensing member 120 comprises a built-in sensor unit 122. The built-in sensor unit 122 may include, but is not limited to, at least one radar sensor for detecting a moving or stationary object in the area 101, at least one motion sensor for detecting a movement in the area 101, at least one acoustic sensor for detecting an acoustic wave in the area 101, at least one timer for timing the operation state of the lighting member 112, at least one temperature sensor for detecting temperature and/or temperature change of the area 101, or a combination of them. In some embodiments, the at least one radar sensor comprises a frequency modulated continuous wave (FMCW) radar. In some embodiments, the at least one motion sensor comprises one or more microwave sensors, and/or one or more passive infrared (PIR) sensors.

In some embodiments, the built-in sensor unit 122 may further include a radio frequency (RF) receiver coupled with the MCU 130 for operably detecting the instruction from the remote operation of the remote control 160.

In one embodiment, the built-in sensor unit 122 may also have a wireless networking module coupled with the MCU 130 for operably detecting the instruction from the remote operation of the APP of the mobile device 160. In one embodiment, the wireless networking module comprises a Bluetooth® module, and/or a Wi-Fi module. The Bluetooth® module enables short-range wireless communication between the germicidal device 100 and other nearby electronic devices such as a computer, a smartphone, a television, a car, etc., nearby the germicidal device 100, while the Wi-Fi module enables the wireless connection of the germicidal device 100 to the internet.

In one embodiment, the sensing member 120 may further include a remote sensor unit 125. The remote sensor unit 125 includes, but is not limited to, an entrance sensor, a photo eye system, or an electric fencing system for detecting an intrusion into the area 101. For example, the electric fencing system may generate an invisible electric fence 150 surrounding the germicidal device 100. When a person or animal approaches to the electric fence 150, the electric fencing system detects the intrusion of the person or animal into the area 101, which in turn, triggers the MCU 130 to turn off the lighting member 112. Similarly, the photo eye system uses infrared sensors to detect such intrusion when a person or animal approaches to the invisible infrared fence generated by the photo eye system. Also, when a person or animal enters a door of the area (room) 101, the entrance sensor can detect the entrance (i.e., intrusion), which in turn, can trigger the MCU 130 to turn off the lighting member 112.

In addition, in certain embodiments, the fence 150 can be created by a built-in sensor such as a microwave sensor, a sound sensor, or others, or the remote sensor such as a door sensor for detecting a closed or open door, entrance sensor for detecting human entering in or out, or other type. When operating in an illumination model, the germicidal device 100 relies on the built-in sensor or remote sensor or both to monitor human or animal movement and automatically shut off the lighting member 112, upon detection by the sensor or tripping of the fence 150.

In certain embodiments, the germicidal device 100 may have three operations modes including a default operation mode, a manual operation mode and a remote operation mode.

When the germicidal device 100 operates in the default operation mode, the lighting member 112 is automatically in the on-state when the time is in the on-state time period, or in the off-state when the time is in the off-state time period. The on-state time period and the off-state time period are usually predetermined/pre-programmed. For example, the on-state time period can be set, but not limited to, from 9:00 AM to 17:00 PM when a room user is away for working, while the off-state time period is set, but not limited to, from 17:00 PM to 9:00 AM when the room user is back home. As such, the lighting member 112 is turned in the on-state and emits the UVC light during 9:00 AM-17:00 PM, and the lighting member 112 is turned in the off-state and does not emit the UVC light during 17:00 PM-9:00 AM. Any time during the on-state time period, when the sensing member 120 detects a movement in the area 101, an expiration of the on-state time period, or a remote instruction to turn off the lighting member 112, the MCU 130 switches the lighting member 112 from the on-state to the off-state. Similarly, any time during the off-state time period, when the sensing member 120 detects an expiration of the off-state time period, or a remote instruction to turn on the lighting member 112, the MCU 130 switches the lighting member 112 from the off-state to the on-state. It should be appreciated that the on-state time period and the off-state time period can be set in other forms, such as in multiple durations.

When the germicidal device 100 is in the remote operation mode remotely operated by a remote control and/or a mobile device 160, the lighting member 112 is operably in one of the on-state and the off-state in response to operations of the remote control 160 or executions of an APP of the mobile device 160. Similarly, any time during the on-state time period, when the sensing member 120 detects a movement in the area 101 or a remote instruction to turn off the lighting member 112, the MCU 130 switches the lighting member 112 from the on-state to the off-state, and any time during the off-state time period, when the sensing member 120 detects a remote instruction to turn on the lighting member 112, the MCU 130 switches the lighting member 112 from the off-state to the on-state. The remote operation mode can avoid UVC light exposure to the user when operating.

When the germicidal device 100 operates in the manual operation mode having an on-operation and an off-operation, a user can initiate the on-operation to turn the lighting member 112 in the on-state to emit the UVC light, and the user can also initiate the off-operation, when the lighting member 112 is in the on-state, to turn the lighting member 112 in the off-state in which no light is emitted.

In one embodiment, the remote operation overrides the default operation, and the manual operation overrides both of the default operation and the remote operation. For example, when the germicidal device 100 is in the default operation mode, a user can remotely turn the lighting member 112 from the on-state to the off-state, or from the off-state to the on-state. When the germicidal device 100 is in the default operation mode or the remote operation mode, a user can manually turn the lighting member 112 from the on-state to the off-state, or from the off-state to the on-state.

In another embodiment, the manual operation overrides the default operation, and the remote operation overrides both of the default operation and the manual operation. For example, when the germicidal device 100 is in the default operation mode, a user can manually turn the lighting member 112 from the on-state to the off-state, or from the off-state to the on-state. When the germicidal device 100 is in the default operation mode or the manual operation mode, a user can remotely turn the lighting member 112 from the on-state to the off-state, or from the off-state to the on-state.

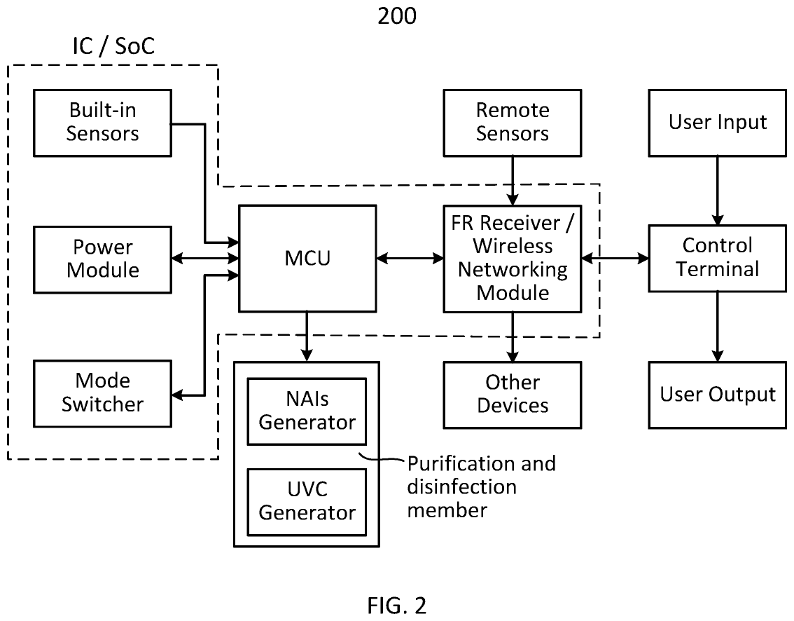

Referring to FIG. 2, a germicidal device 200 is schematically shown according to another embodiment of the invention. Similar to the germicidal device 100 shown in FIG. 1, the germicidal device 200 includes the purification and sterilization member an UVC lighting member, a sensing member including built-in sensors and remote sensors, an RF receiver, a wireless networking module and an MCU. The purification and sterilization member in one embodiment includes an UVC generator (lighting member) and a NAIs generator.

The germicidal device also includes a power module coupled with the MCU for providing power to the lighting member, the sensing member and the MCU. The power module may comprise a power driver circuit and a battery coupled to the power driver circuit. The battery may be a rechargeable battery. In this case, the power module further comprises a wireless charging module coupled to the power driver circuit for wirelessly charging the rechargeable battery.

The germicidal device may further include a model switch coupled to the MCU for switching the operation modes of the germicidal device.

In certain embodiments, some or all of the built-in sensors, the power module, the model switch, the RF receiver, the wireless networking module and the MCU are integrated into a system on chip (SoC), or a single integrated circuit (IC), or two or more ICs. In certain embodiments, the wireless networking module such as the Wi-Fi module and/or the Bluetooth® module may include its own controller, which is coupled to the MCU. The MCU in certain embodiments is configured to control the operations of the germicidal device.

The operations and functions of the germicidal device 200 are similar to that of the germicidal device 100. (1) Sensor responses: the sensor member (built-in and remote sensor) collects information and sends it to the MCU. The MCU commands the power module or the mode switcher to change the operation state or shut down. At the same time, the MCU can send data to a network and the cloud via the wireless networking module. Other devices in the same network can read the data and do the response actions like shut down itself if in the same room. The terminal can receive the data, too, though the Internet/Wi-Fi. The terminal can be a smartphone, a personal computer (PC), an IPC (industrial PC), or the likes. The processing center can inform the user or administrator of the device's operations or ask the user to perform the operations. (2) Automatic run based on the user input, e.g., a space type such as an elevator or a bathroom, etc., the area size, and so on. The terminal can transfer such input into operation parameters informing the sensor and the MCU about optimal run-time and logic model or an electric fence range, etc. (3) Remote and central control: the user can remotely control the device (start-up, shutdown, and modes) via the network. (4) Remote monitoring: the user or monitor software can monitor the device via the network, and provide information on the running status, the remaining lifetime, and other statistics (big data). (5) Remote sensors network: the remote sensors can join in the network and send the data such as motion detection to the network. The devices and the terminals can receive and perform the responsive actions. For example, before a human enters into an elevator, the sensor outside the elevator detects the human's entrance and sends that data to the UV germicidal device to change the operation state or shut down.

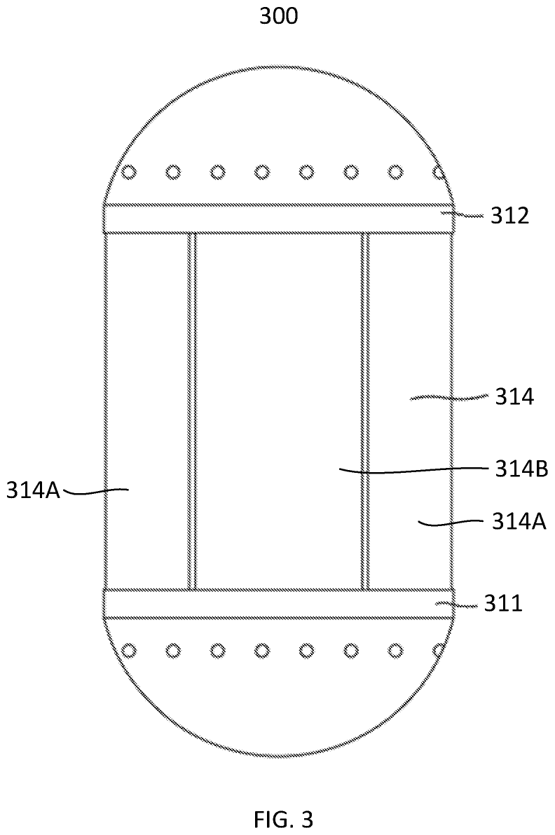

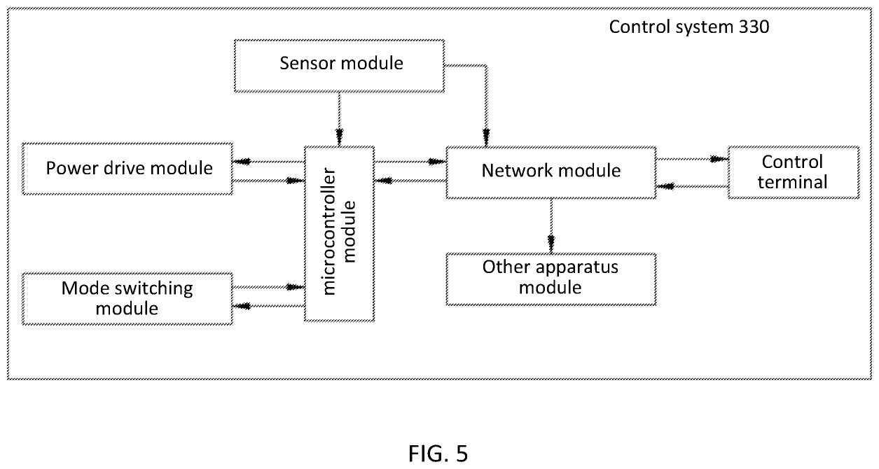

FIGS. 3-5 show a germicidal device 300 according to one embodiment of the invention. The germicidal device 300 is a design for a stand-alone use. Similar to the germicidal device 100 shown in FIG. 1, the germicidal device 300 includes a purification and sterilization member 320 placed inside a housing structure 310, and a control system 330 (FIG. 5).

The housing structure 310 includes a base 311, a top cover 312, support rods 313 connecting the base 311 and the top cover 312, a casing 314 detachably attached to the base 311 and the top cover 312, at least one air inlet 315 formed on one side of the base 311, and at least one air outlet 316 formed on one side of the top cover 312. The air inlet 315 and the air outlet 316 in one embodiment are kept on the same vertical plane. It should be appreciated that the air inlet and the air outlet can also be formed in other portions of the housing structure 310.

In some embodiment, the number of the connecting rods 313 is set to six. The six connecting rods 313 are distributed on the base 311 in a circle. The connecting rods 313 are used to connect the base 311 and the top cover 312. It should be appreciated other numbers of connecting rods with other arrangements can also be utilized to practice the invention.

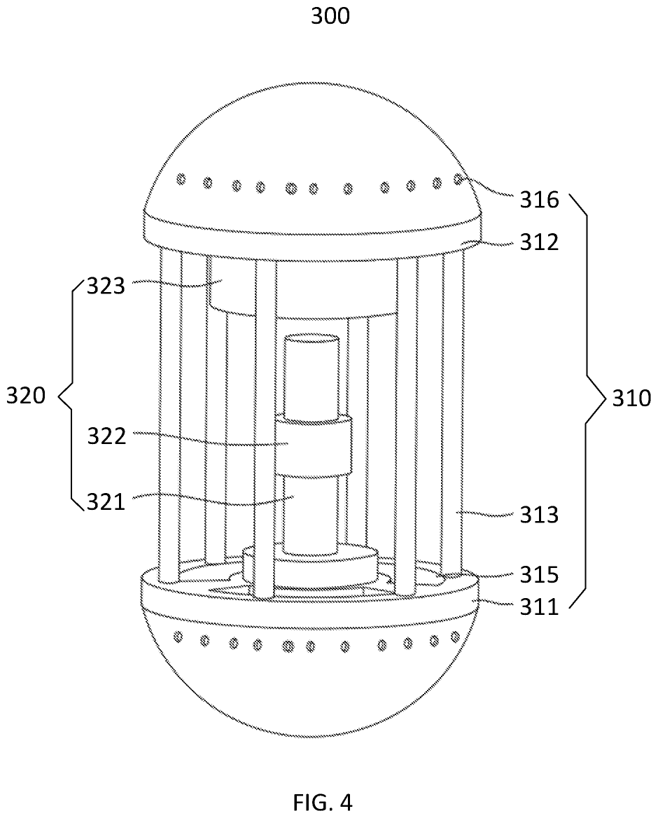

The purification and sterilization member 320 is arranged inside the housing structure 310, that is, the purification and sterilization member 320 is enclosed by the housing structure 310. The purification and sterilization member 320 includes an negative air ions generator 321, a UVC generator 322 and a circulating member 323 such as the suction fan. The negative air ion generator 21 and the UVC generator 22 can emit respectively NAIs and a narrow-spectrum light beam with a wavelength of about 100-400 nm or a combination of thereof. The negative air ions generator 321 is fixed on the UVC light generator 322, the UVC light generator (lighting member) 322 is fixed above the base 311, and the suction fan 323 is located directly above the UVC generator 322, as shown in FIG. 4.

In one embodiment, the housing structure 310, as an external component of the germicidal device 300, is in the shape of a cylinder or a semi-elliptical cylinder, or other shapes, small in size, light in weight, easy to carry, and easy to use. It should be appreciated that the housing structure 310 can also formed in other forms.

In one embodiment, the shape of the casing 314 is designed to be cylindrical. The casing 314 comprises a plurality of shutter boards 314A, 314B, 314C, . . . that are movably jointed or fixed to each other. In addition, the casing 314 can be rotated automatically. In one embodiment, the casing 314 is operably switchable in an open state or a close state. When in the open state (FIG. 4), the shutter boards 314A, 314B, 314C, . . . are moved to define one or more windows for allowing the UVC light emitted from the lighting member (UVC light generator) 322 to pass through the one or more windows to illuminate said space (area). When in the close state (FIG. 3), the shutter boards 314A, 314B, 314C, . . . are moved to close the one or more windows for circulating air through the air pass-through channel defined between the air inlet 315 and the air outlet 316. Accordingly, the germicidal device 300 can selectively operate in a light disinfection mode in which the casing 314 is in the open state, or in an air purification mode in which the casing 314 is in the close state.

In addition, the casing 314 comprises holes that are easy for the NAIs and the UVC light to disinfect and purify the air by natural convection or use the fan 323 to strengthen the air circulation to enhance the air purification and sterilization function.

The purification and sterilization member 320 mainly plays the role of purifying and sterilizing the air, wherein the UVC generator 322 can be an ozone-free UVC lamp or an ozone UVC lamp. The purification and sterilization member 320 in some embodiments can include functions of light and sound sensors, a Wi-Fi module, a Bluetooth module, USB and direct wall sockets. The purification and sterilization member 320 in some embodiments includes a low-pressure mercury lamp formed by the combination of the negative air ion generator 21 and/or the low-pressure discharging lamp 322 generating 185 nm wavelength light or 253.7-254 nm wavelength quasi-molecular discharging generated 222 nm wavelength light. i.e., UVC light generator 322 with two main emission lines: one line is about 222 nm or 253.7 nm wavelength, the other line is a 185 nm wavelength, both of which are invisible to the naked eye. The UVC light of the 222 nm or 253.7 nm wavelength can sterilize the surface of the object, while UVC light of the 185 nm wavelength can ionize the air to produce ozone, thereby sterilizing the air. Ozone has a strong oxidative and efficient air sterilization effect, which can overcome the shortcomings of ultraviolet rays that only spread in a straight line so as to cause disinfection dead zones.

In some embodiments, the high-efficiency negative air ion generator 321 in the purification and sterilization member 320 is used to effectively remove the odor and dust in the air through the continuously generated NAIs after the germicidal device 300 is power-on, thereby increasing the content of negative ions and oxygen, effectively purifying the environment, and enhancing the overall efficiency of the purification and sterilization member 320.

Table 1 lists the NAIs test of the germicidal device with an input voltage of 110 V for different output voltages.

TABLE 1
NAIs test report

| No. | Output Voltage | Amount of NAIs | Input Current |
|---|---|---|---|
| 1 | −3.10 KV | 3600 W | 3.0 mA |
| 2 | −3.15 KV | 3560 W | 3.0 mA |
| 3 | −3.30 KV | 3650 W | 3.0 mA |
| 4 | −3.20 KV | 3450 W | 3.0 mA |
| 5 | −3.30 KV | 3500 W | 3.0 mA |
| 6 | −3.40 KV | 3640 W | 3.5 mA |
| 7 | −3.20 KV | 3300 W | 2.0 mA |
| 8 | −3.21 KV | 3550 W | 3.0 mA |
| 9 | −3.35 KV | 3600 W | 3.0 mA |
| 10 | −3.40 KV | 3680 W | 3.0 mA |
| Ave. | | 3553 W | |

Table 1 is the results from 10 test for our NAIs generator, When the Output voltage is −3.10 KV and the input current is 3.0 mA, it can generate 36 million negative ions. The average Output Voltage is −3.261 KV and the average Input current is 2.95 mA, it can generate 35.53 million negative ions. The World Health Organization regards the content of “negative oxygen ions” in the air as one of the important indicators to measure whether the air quality is fresh. The World Health Organization stipulates that the standard concentration of negative ions in fresh air should be greater than 1500/cm³; an environment with a concentration of 4000/cm³ has health care effects.

By setting up of the purification and sterilization member with the negative air ion generator and the UVC generator together, the NAIs generated can effectively remove odor and dust in the air, such as PM2.5. aerosols, mold spores, pollen, etc., thereby achieving high-efficiency air purification effect. At the same time, after the device continues to work, it can also effectively increase the content of negative ions and oxygen in the air, further improve air quality. In addition, the high UVC generated by the dual-wavelength UVC lamps and the UVC excited ozone can kill pathogens including, but are not limited to, new coronavirus, mites, bacteria, molds, fungi, etc., in the air and surfaces within the effective range without dead ends, so that all indoor objects can be effectively sterilized and disinfected, thereby realizing efficient disinfection and effectively avoiding bacterial infection caused by human contact with objects, making the efficacy of disinfection device more comprehensive, providing people with a healthy living environment, and reducing the harm caused by bacteria in the space to the human body.

The intelligent control system 330 can be configured to control UVC light generator 322 within a safe range to ensure that users are not harmed by UVC light and ozone. In addition, there is no noise and no filter in the germicidal device.

As shown in FIG. 5, the control system 330 includes a sensor module (member), a microcontroller module (unit), a power drive module, a mode switching module, a network module, a control terminal and other device terminals.

The sensor module is used to sense changes in the external environment of the germicidal device in real time. The sensor module includes one or both of a built-in sensor and an external sensor. The built-in sensor includes a microwave sensor, a sound sensor, and/or an infrared sensor. The external sensor may include remote sensors and/or entrance sensors.

The microcontroller module is configured to receive the information sent by the sensor module, and control the operation of the power drive module and the mode switching module.

The network module is configured to receive the data sent by the microcontroller module, and further configured such that the other devices in the same network can read the data and perform corresponding operations. The control terminal can be controlled by a smartphone, a personal computer or an IPC, or any one or more of them.

The mode switching module is configured to switch between the air purification mode and the light disinfection mode of the germicidal device. The power drive module is used for providing power support for the germicidal device.

In operations of the control system 330, the sensor module collects information and sends it to the microcontroller module of the germicidal device, and the microcontroller module can instruct the power drive module and the mode switching module to change the mode or shut down. At the same time, the microcontroller module can send data to the network and the cloud, and other devices in the same network can read the data and perform response operations, such as shutting down itself in the same room. The control terminal can also receive data through Wi-Fi or Bluetooth, and the control terminal can be smart phone, personal computer or IPC, etc. The processing center of the system can notify the user or administrator of the operations of the germicidal device, or require the user to perform an operation. The user can operate the control terminal to realize the automatic operation of the user input, for example, such as an elevator or bathroom, etc., and the control terminal can convert these inputs into operating parameters to inform sensor modules and microprogrammed control modules of optimal runtime and logic model (or range of electric fence), etc. In addition, the user can remotely control the device (start, stop and mode) through the network, thereby realizing remote and central control. The user or monitoring software can monitor the germicidal device through the network, and provide information about the operating status, remaining life and the information of other statistical results (big data) can be monitored remotely. The control system can also add remote sensors to the network, and send data (such as motion detection data) to the network to realize remote sensor network functions.

It is well known that the SARS-CoV-2 virus is sensitive to the UVC light. According to the invention, the intelligently controlled purification and sterilization (germicidal) device, as marketed as Inoculight®, can emit UVC light with wavelengths of 222 or 253.7 nm and/or 185 nm, which can effectively kill the SARS-CoV-2 virus. In addition, it also effectively purifies the environment such as odor and dust in the air through high-efficiency NAIs, increases the content of negative ions and oxygen in the air, and integrates intelligent wide-area contact sensing, Wi-Fi distribution network App remote control, Bluetooth distribution network APP control, thereby purifying air and eliminating viruses and protecting users from UVC damage.

In certain embodiments, the intelligently controlled purification and sterilization device can be used to purify indoor public places such as homes, kitchens, classrooms, student dormitories, hotel rooms, restaurant clubs, entertainment places, etc., in cars, elevators, etc. It can also be used to purify small appliances such as mobile phones and refrigerators.

In certain embodiments, the intelligently controlled purification and sterilization device is a portable device, namely a portable Inoculight®, which includes a portable air purifier and sterilizer, has human-sensing and remote control functions and can be used anytime, anywhere for purification and sterilization of the environment.

In certain embodiments, the intelligently controlled purification and sterilization device is a miniature device, which can generate negative air ions and 100-400 nm UVC light; and is compatible with a wide range of voltages (110-240V, 3.3-18V), standard and/or USB power connectors, built-in battery (optional); wall-mounted power supply installation, attached installation, ceiling installation and other installation methods, human body induction and timing control and Wi-Fi/Bluetooth distribution network APP control and cloud data storage.

In certain embodiments, the intelligently controlled purification and sterilization device includes, but is not limited to, the following operations: simply plugging the device into a wall outlet, or charging the battery, and placing the device where it is to be used, i.e., one can remotely control timing, turn on, turn off, and human body induction through the mobile app, automatically turn on the light when a person or pet leaves, and automatically turn off the light when a person or pet enters.

Through the housing structure 310, the purification and sterilization member 320 and the control system 330 are used together, so that the device combines intelligent technology and space, surface and air purification and sterilization functions to ensure sensing of people movements and intelligent disinfection, which not only results in effective purification and sterilization effects in the indoor environment, but can also avoid UVC damage to the human body, which is further improvements to the traditional single UVC or air purification device. Compared with a traditional device, the intelligently controlled purification and sterilization device of the invention only needs to be directly inserted into the power socket, has zero noise, zero consumables, low power consumption, small size, easy to carry, cost optimization through tools such as value engineering, making the product cheap and cost-effective. In addition, through the intelligent adaptive functions, it can be automatically applied to different scenarios and has a wide range of applications. It can be used for indoor (such as hallway, corridor, bedroom, living room, shoe cabinet, kitchen, bathroom, student dormitory, classroom, office, hotel room, etc.), elevators, cars, small devices (mobile phones, iPads), restaurants and other public places, etc.

In certain embodiments, the intelligently controlled purification and sterilization device can be operated as follows:

First, the user sets the operation mode of the device through the control terminal, and the control terminal converts the user input commands into operating parameters, and sends them to the sensor module and the microcontroller module with the optimal running time and logic model.

When the user sets the device to run the air purification mode, the negative air ion generator 321, the UVC generator 322 and the suction fan 323 are activated at the same time, and the suction fan 323 runs so that the air outside the device can quickly enter the device through the air inlet 315. Inside the device, the UVC light and ozone generated by the UVC generator 322 and the negative air ion generator 321 irradiate the air and the surface of the object, so as to kill germs and viruses in the air. At the same time, the NAIs generated by the negative air ion generator 21 increase the oxygen concentration in the environment, thereby improving the indoor air quality, and at the same time, it can also reliably sterilize and purify the air by natural convection or use a fan to strengthen the air circulation to accelerate the air purification and sterilization function.

If the device is set to operate in the lighting disinfection mode, the microcontroller module controls the casing 314 to be in the open state, so that the UVC generated inside is no longer obstructed by the casing 314, and can be directly irradiated into the external environment, thereby enabling external disinfection and sterilization of objects.

When someone enters the room, the movement signal is detected by the sensor module and sent to the microcontroller module. The micro controller module selects and executes two protection measures. One is to control the power drive module to turn off, so that the entire device stop working, the second is to control the casing 314 to be closed to keep it in the close state, and then continue to carry out air disinfection inside the n device. When the person leaves, the device will continue to work again.

When the user needs to operate the device, by operating the control terminal, it can access the network module, directly read the relevant data inside it, and can perform startup, shutdown, timing and operation mode switching respectively. It can also monitor the running status of each component inside the device in real time.

It should be noted that all or a part of the steps according to the embodiments of the present invention is implemented by hardware or a program instructing relevant hardware. Yet another aspect of the invention provides a non-transitory tangible computer-readable medium storing instructions which, when executed by one or more processors, cause a germicidal device to perform the above-disclosed method for killing microorganisms in an area. The computer executable instructions or program codes enable a computer or a similar computing system to complete various operations in the above disclosed method for privilege management. The storage medium/memory may include, but is not limited to, high-speed random access medium/memory such as DRAM, SRAM, DDR RAM or other random access solid state memory devices, and non-volatile memory such as one or more magnetic disk storage devices, optical disk storage devices, flash memory devices, or other non-volatile solid state storage devices.

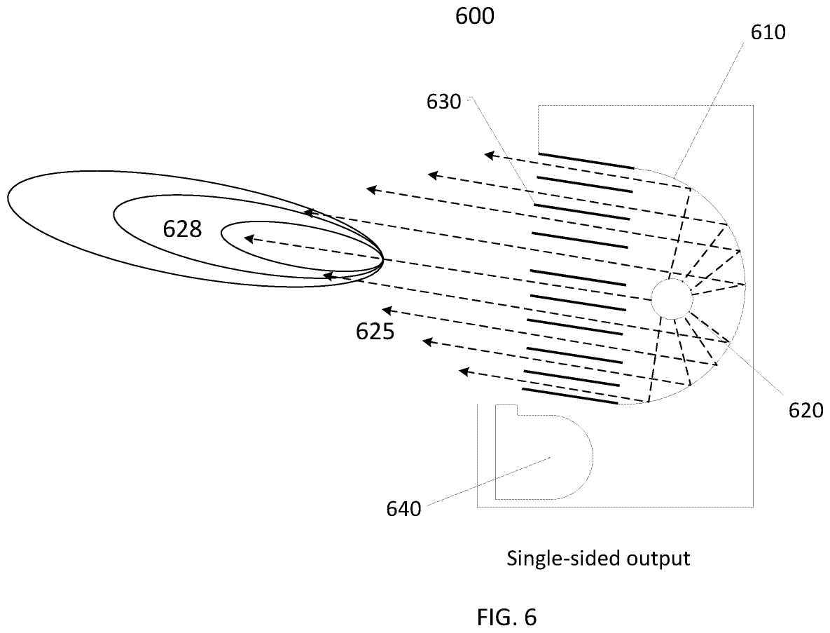

Referring to FIG. 6, one embodiment of the intelligently controlled purification and sterilization device 600 is shown according to the invention. The intelligently controlled purification and sterilization device 600 is a single-side output device that includes a UVC light generator 620 for emitting the UVC light, and an air circulation and/or ion generation device 640 for circulating air and generating NAIs. The intelligently controlled purification and sterilization device 600 further includes a light reflection member 610 with high reflectivity configured to collect the UVC light 625 emitted from the UVC light generator 620, and a grating member 630 configured to direct the collected UVC light 625 into a particular direction to sterilize and purify the entire surface and space. The grating member 630 may include a plurality of bearing plates being parallel to each other, so as to adjust the UVC light 625 output in parallel. In addition, the angle of the plurality of bearing plates is adjustable so that the UVC light 625 output can be directed to the desired direction.

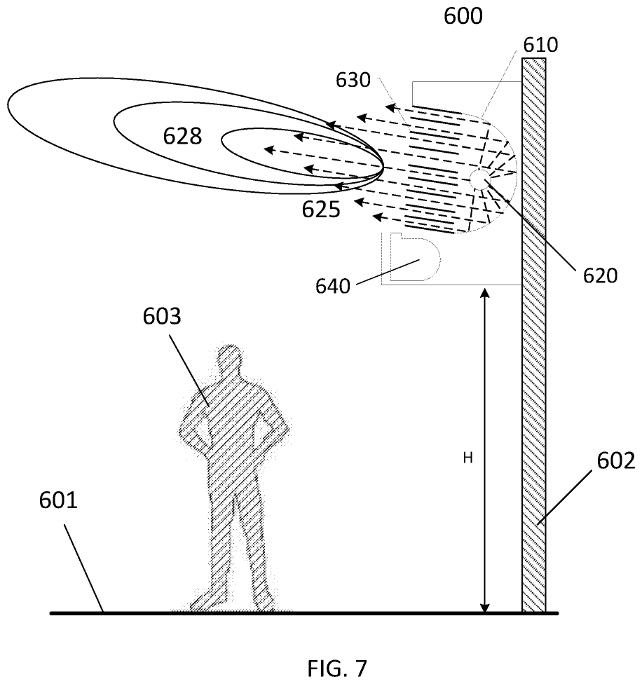

In some embodiments, the high-intensity UVC light of 100-400 nm is generated by the power-driven LED UVC light generator 620. The high-intensity UVC light 625 is collected to a specific angle or surface together with the reflective member 610, and after the precise adjustment of the grating member 630, the UVC light 625 forms a high-intensity beam to sterilize and purify the entire surface and space in a germicidal zone 628. As shown in FIG. 7, in use, the intelligently controlled purification and sterilization device 600 can be attached to a post or wall 602 in a position having a height of H from ground. The height of H can be higher than a human body 603. Accordingly, the light 625 of the intelligently controlled purification and sterilization device 600 is output in parallel into the germicidal zone 628 (large-area UVC light layer, or upper layer) that is higher than the human body. The large-area UVC light layer (germicidal zone) 628 is farther away from the human body, safer and more uniform, so that the disinfection surface is wider, and the disinfection effect is more effective. At the same time, it cooperates with the circulating fan and negative air ions generating device 640 to purify and disinfect the air.

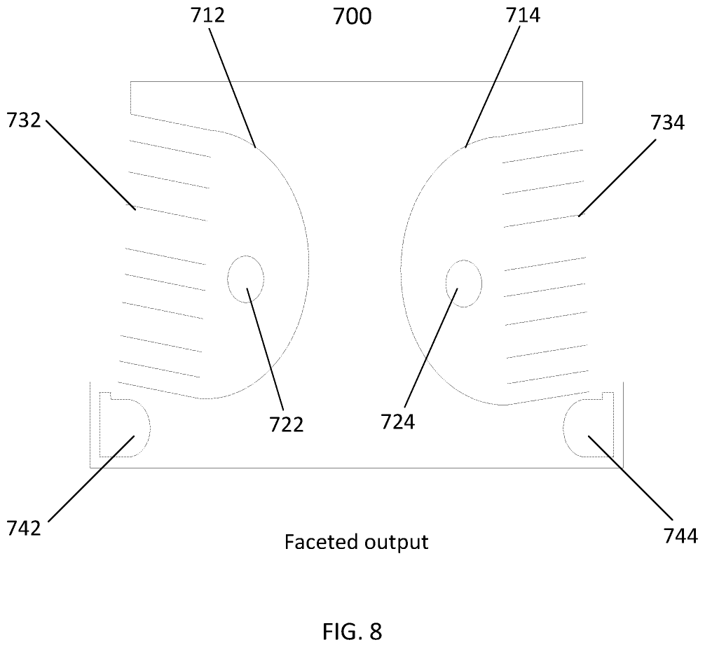

Referring to FIG. 8, another embodiment of the intelligently controlled purification and sterilization device 700 is shown according to the invention. The intelligently controlled purification and sterilization device 700 is similar to the device 600 shown in FIG. 6, except that the device 700 is a faceted output device that includes two UVC light generators 722 and 724; two air circulation and/or ion generation devices 742 and 744; two reflective members 712 and 714; and two grating members 732 and 734, arranged in a pair-imaged form of two devices 600, so that the UVC light can be simultaneously output to spaces/areas from two opposite sides of the device for the purification and sterilization.

Among other things, the invented devices can be used in places such as theaters, airports, railway stations, warehouses, food production lines, logistics sorting lines, conference rooms and various large-scale personnel-intensive places, etc.; public transportation such as school buses, medical ambulances, buses, ambulances, airplanes, etc.; and logistics transportation such as material transport ships, vehicles, vehicle-mounted cold chains, transport aircraft, field dining vehicles, import and export transport vehicles, etc.

According to the invention, the intelligently controlled purification and sterilization device has the following beneficial effects:

By setting the purification and sterilization member 320 and cooperating with the housing structure 310, the device has two modes of use at the same time, which can realize the purification of indoor air and the disinfection of external objects. The germs on indoor objects can be directly irradiated through the combination of the negative air ion generator 321 and the UVC generator 322. With its excellent disinfection and sterilization effects, the objects can be efficiently purified, thereby effectively preventing the human body from contacting with objects and cause bacterial infection, making the disinfection device more comprehensive, providing people with a healthy living environment, and reducing the damage caused by bacteria in the space to the human body.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

What is claimed is:

1. A germicidal device, comprising:
a purification and sterilization member configured to generate UVC light and negative air ions (NAIs) for destroying microorganisms and removing odors in a space in which the purification and sterilization member is placed, wherein the UVC light having one or more wavelengths in a range of about 100-400 nm;
a sensing member configured to detect at least one event occurred in said space in real time, wherein the at least one event includes a movement in said space, an acoustic wave in said space, an intrusion into said space, an expiration of an on-state time period in which the UVC light is generated, an expiration of an off-state time period in which the UVC light is not generated, and/or an instruction from a remote operation; and
a microcontroller unit (MCU) coupled with the purification and sterilization member and the sensing member for controlling operations of the purification and sterilization member in a respective state in accordance with the at least one event occurred in said space.

2. The germicidal device of claim 1, further comprising a housing structure comprises a base, a top cover, support rods connecting the base and the top cover, a casing detachably attached to the base and the top cover, at least one air inlet formed on the base, and at least one air outlet formed on the top cover, wherein the purification and sterilization member is fixed on the base so as to be enclosed by the housing structure.

3. The germicidal device of claim 2, wherein the at least one air inlet and the at least one air outlet are arranged on a same vertical plane.

4. The germicidal device of claim 2, wherein the casing comprises a plurality of shutter boards that are movably jointed or fixed to each other.

5. The germicidal device of claim 4, wherein the casing is operably switchable in an open state or a close state, wherein in the open state the shutter boards are moved to define one or more windows for allowing the UVC light emitted from the lighting member to pass through the one or more windows to illuminate said space, wherein in the close state the shutter boards are moved to close the one or more windows for circulating air through at least one air pass-through channel defined between the at least one air inlet and the at least one air outlet.

6. The germicidal device of claim 5, being configured to selectively operate in a light disinfection mode in which the casing is in the open state, or in an air purification mode in which the casing is in the close state.

7. The germicidal device of claim 2, further comprising a circulating member that is located directly above the purification and sterilization member for operably circulating air.

8. The germicidal device of claim 7, wherein the circulating member comprises one or more suction fans.

9. The germicidal device of claim 1, wherein the purification and sterilization member is configured to generate the UVC light has a wavelength of about 185 nm, 222 nm or 253.7 nm, or two wavelengths with one at about 185 nm and the other at about 222 nm or 253.7 nm.

10. The germicidal device of claim 1, wherein the purification and sterilization member an negative air ions (NAIs) generator configured to generate the negative air ions (NAIs), and a lighting member configured to generate the UVC light.

11. The germicidal device of claim 10, wherein the lighting member comprises one or more UVC light-emitting diodes (LEDs), an ozone-free UVC lamp, an ozone UVC lamp, or a low-pressure mercury lamp.

12. The germicidal device of claim 1, wherein the sensing member comprises a built-in sensor unit comprising at least one radar sensor for detecting a moving or stationary object in said space, at least one motion sensor for detecting a movement in said space, at least one acoustic sensor for detecting an acoustic wave in said space, at least one timer for timing the operation state of the lighting member, at least one temperature sensor for detecting temperature and/or temperature change of said space, or a combination of them.

13. The germicidal device of claim 12, wherein the at least one motion sensor comprises one or more microwave sensors, and/or one or more passive infrared (PIR) sensors.

14. The germicidal device of claim 12, wherein the built-in sensor unit further comprises a radio frequency (RF) receiver coupled with the MCU for operably detecting the instruction from the remote operation of the remote control, and/or a wireless networking module coupled with the MCU for operably detecting the instruction from the remote operation of the APP of the mobile device.

15. The germicidal device of claim 14, wherein the wireless networking module comprises a Bluetooth® module, and/or a Wi-Fi module.

16. The germicidal device of claim 12, wherein the sensing member further comprises a remote sensor unit including an entrance sensor, a photo eye system, or an electric fencing system for detecting an intrusion into said space.

17. The germicidal device of claim 1, further comprising a power module for providing power to the lighting member, the sensing member and the MCU.

18. The germicidal device of claim 1, further comprising at least one light reflection member configured to collect the UVC light, and at least one grating member configured to direct the collected UVC light into a particular direction to sterilize and purify the entire surface and space.

19. The germicidal device of claim 1, being configured to operate in one of:
a default operation mode in which the purification and sterilization member is automatically in an on-state when the time is in an on-state time period, or in an off-state when the time is in an off-state time period, wherein the on-state time period and the off-state time period are predetermined, and wherein the purification and sterilization member emits the UVC light in the on-state, or no light in the off-state;
a manual operation mode having an on-operation and an off-operation, wherein the on-operation causes the lighting member to be in the on-state, and the off-operation causes the lighting member to be in the off-state; and
a remote operation mode in which the purification and sterilization member is operably in one of the on-state and the off-state in response to operations of a remote control or executions of an APP of a mobile device.

20. The germicidal device of claim 19, being configured such that
when the purification and sterilization member is in the on-state, one of a movement in said space, an expiration of the on-state time period and a remote instruction to turn off the purification and sterilization member detected by the sensing member causes the MCU to switch the purification and sterilization member from the on-state to the off-state; or
when the lighting member is in the off-state, an expiration of the off-state time period or a remote instruction to turn on the purification and sterilization member detected by the sensing member causes the MCU to switch the purification and sterilization member from the off-state to the on-state.
